(19) United States
(12) Patent Application Publication
(10) Pub. No.: US 2017/0252181 A1
(43) Pub. Date: Sep. 7, 2017

(54) METHOD OF IMPLANTING A CORPECTOMY CAGE

(71) Applicants: Frank Acosta, Los Angeles, CA (US); Dave Wells, Santa Monica, CA (US)

(72) Inventors: Frank Acosta, Los Angeles, CA (US); Dave Wells, Santa Monica, CA (US)

(21) Appl. No.: 15/059,024

(22) Filed: Mar. 2, 2016

(51) Int. Cl. A61F 2/44

(52) U.S. Cl. CPC A61F 2/4455; A61F 2002/4475; A61F 2220/0016; A61F 2220/0033; A61F 2230/0021; A61F 2230/0026; A61F 2230/0069

(57) ABSTRACT

Embodiments of the present disclosure include a method for implanting a corpectomy cage within a patient's intervertebral cavity space during a surgical corpectomy procedure. The method may include removing a diseased vertebra of a patient from a target incision site to create an intervertebral cavity space within a vertebral column. The method may also include selecting a central core with a height suited to be securely fitted within the intervertebral cavity space to restore the height of the vertebral column. In some embodiments, the method proceeds to selecting a first endcap that matches the patient's existing spinal configuration and anatomy to attach to a first end of the central core. Next, the corpectomy cage may be assembled by attaching a first surface of the first endcap to the first end of the central core. In some embodiments, the method may proceed to packing bone material into a cavity formed within the first endcap, the cavity having a cavity floor to contain the bone material. Next, the corpectomy cage may be implanted within the intervertebral cavity space to promote a fusion between the bone material and a vertebra at one end of the intervertebral cavity space.

### TECHNICAL FIELD

The disclosed technology relates generally to an implant device. More specifically, the present invention relates to a vertebral implant device used during a corpectomy procedure to reconstruct the spine.

### BACKGROUND

A patient suffering from severe spinal cord injuries resulting from trauma, tumors, or spinal column deformity may lead to serious conditions where pressure is placed on the spinal cord and pinches the nearby nerve roots. In such instances, a surgical procedure may be required to remove the diseased and damaged parts of the vertebral bone in order to effectively relieve pressure on the spinal cord and the nearby spinal nerves.

FIG. 1 (prior art) illustrates an exemplary corpectomy device 110, or corpectomy cage 110, implanted within an intervertebral cavity 115 that once contained the one or more diseased or injured vertebra. A corpectomy procedure to implant the corpectomy device 110 can be performed on any part of the spine 105, such as the cervical, thoracic, or lumbar spine. At the onset of the surgical procedure, the diseased or injured vertebra is first removed, along with any of the remaining diseased vertebral fragments. The vertebral empty space, or intervertebral cavity space 115, is then further widened to receive a corpectomy cage 110 so that the device may act as a bone substitute to occupy the intervertebral cavity space 115. Upon secure placement of the corpectomy cage 110 within the intervertebral cavity space 115, the corpectomy cage 110 may then provide proper spinal support.

However, one of the most common risks associated with a corpectomy procedure is that the vertebra in contact with the inserted corpectomy cage 110 may fail to undergo proper spinal fusion, such that the inserted corpectomy cage 110 is no longer able to stably position or support the spine in its proper upright position. At such an instance, nearby tissue may be harmed as a result of the shifting corpectomy cage, which then likely requires additional surgery to reposition and securely reattach the corpectomy cage 110 to the spine. In other cases, a new corpectomy cage 110 altogether must be implanted. Additional surgery not only prolongs the recovery process, but also significantly increases the risk of infection, nerve damage, and spinal cord damage.

### BRIEF SUMMARY OF EMBODIMENTS

In light of the above-described problems associated with implanting a corpectomy cage, there is a need for a corpectomy cage that provides enhanced stability when positioned and aligned within a patient's spine. Additionally, there is a further need for a corpectomy implant device to further enhance the union or fusion with the vertebra above and below the intervertebral cavity space, so that the corpectomy cage is properly stabilized and fused with the patient's spine.

Embodiments of the disclosed technology are directed towards a method for implanting a corpectomy cage. The process may include removing a diseased vertebra of a patient from a target incision site to create an intervertebral cavity space within the vertebral column. In some embodiments, the process may proceed to selecting a central core with a height suited to be securely fitted within the intervertebral cavity space to restore the height of the vertebral column. Next, the process may proceed to selecting a first endcap that matches the patient's existing spinal configuration and anatomy to attach to a first end of the central core. Next, the process may proceed to assembling the corpectomy cage by attaching a first surface of the first endcap to the first end of the central core. In some embodiments, the process may proceed to packing bone material into a cavity formed within the first endcap, the cavity having a cavity floor to contain the bone material. Next, the process may proceed to implanting the corpectomy cage within the intervertebral cavity space to promote a fusion between the bone material and a vertebra at one end of the intervertebral cavity space.

In some embodiments, the method may include selecting a first endcap and the second endcap such that the endcap has a lordotic angle configured to restore the natural inward lordotic curvature of a spine. By way of example only, the lordotic angle of the endcap may have a range from 1 to 11 degrees. Additionally, the first endcap may have a second surface configured to engage with the vertebra at one end of the intervertebral cavity space, such that the first endcap comprises a raised surface protruding from the second surface configured to retard movement of the first endcap when in contact with the vertebra at the intervertebral cavity space. By way of example only, the raised surface protruding from the cavity floor may include at least one of teeth, pointed edges, spikes, mounds, circular columns, rectangular columns, triangular columns, and saw tooth edges.

In some embodiments, the first endcap includes a snap fit attachment located at the first surface, such that the first surface securely interlocks with a corresponding snap fit attachment on the first end of the central core. In some embodiments, the cavity further comprises an opening that passes through the height of the endcap, and the end of the central core has a solid body that is configured to provide a floor to the opening, thereby allowing the cavity to contain the bone material. By way of example only, the bone material may include at least one of bone allograft, bone autograft, osteoinductive agents, demineralized bone matrix, and hydroxyapatite.

In other embodiments, the central core may include a hollow body configured to be packed with bone material. As such, some embodiments may include packing the bone material into the hollow body until both the hollow body and the cavity are completely filled to promote a solid single union of the vertebral column. By way of example only, the bone material may be packed into the hollow body via and opening in the cavity floor. Additionally, the area of the opening of the cavity floor may range from 30-80% of the area of the cavity floor.

In another embodiment, the method for implanting a corpectomy cage may include removing a diseased vertebra from a target incision site to create an intervertebral cavity space within a vertebral column. Next, the process may proceed to selecting an assembled corpectomy cage comprising a central core, with a first endcap attached to a first end of the central core, and a second endcap attached to a second end of the central core. In further embodiments, the process may include packing bone material into a cavity formed within at least one of the first endcap and the second endcap, the cavity having a cavity floor. Next, the process may proceed to implanting the assembled corpectomy cage within the intervertebral cavity space such that the first endcap makes contact with a first vertebra above the intervertebral cavity space, and the second endcap makes contact with a second vertebra below the intervertebral cavity space.

Some embodiments of the corpectomy cage ma include a first endcap with a first surface configured to attach to the central core of the corpectomy cage and a second surface configured to engage with the first vertebra. Additionally, in some embodiments, the first endcap may also include a raised surface that includes at least one of teeth, pointed edges, spikes, mounds, circular columns, rectangular columns, triangular columns, and saw tooth edges to retard a movement of the first endcap when in contact with the vertebra. In other instances, a second endcap may also be attached to the corpectomy cage. In some embodiments, the second endcap may also include a raised surface that includes at least one of teeth, pointed edges, spikes, mounds, circular columns, rectangular columns, triangular columns, and saw tooth edges to retard a movement of the second endcap when in contact with the vertebra.

In some instances, the central core may have a solid body. In other instances, the central core may have a hollow body configured to be packed with bone material. By way of example only, packing bone material into the cavity may include filling the hollow body with the bone material until both the hollow body and the cavity is completely filled with the bone material to promote a single solid fusion of the vertebral column.

Some embodiments may include adjusting the height of the assembled corpectomy cage prior to implanting the assembled corpectomy cage within the intervertebral cavity space. In other embodiments, the process may include adjusting the height of the assembled corpectomy cage after implanting the assembled corpectomy cage within the intervertebral cavity space. By way of example only, the height of the central core of the corpectomy cage may be achieved by using an adjusting mechanism that includes a screw that raises or lowers the height of the central core.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a diagram illustrating a perspective view of a prior art corpectomy cage implanted within a spine of a patient.

FIG. 2A is a diagram illustrating a perspective view of an exemplary endcap for a corpectomy cage having cavities according to certain embodiments of the present disclosure.

FIG. 2B is a diagram illustrating a cross-sectional view of an exemplary endcap for a corpectomy cage having cavities according to certain embodiments of the present disclosure.

FIG. 3A is a diagram illustrating a perspective view of an exemplary endcap for a corpectomy cage having a cavity according to certain embodiments of the present disclosure.

FIG. 3B is a diagram illustrating a cross-sectional view of an exemplary endcap for a corpectomy cage having a cavity according to certain embodiments of the present disclosure.

FIG. 4 is a diagram illustrating a perspective view of an exemplary endcap for a corpectomy cage having a cavity according to certain embodiments of the present disclosure.

FIG. 5 is a diagram illustrating a perspective view of an exemplary endcap for a corpectomy cage having a cavity according to certain embodiments of the present disclosure.

FIG. 6A is a diagram illustrating a perspective view of an exemplary endcap for a corpectomy cage having a cavity according to certain embodiments of the present disclosure.

FIG. 6B is a diagram illustrating a cross-sectional view of an exemplary endcap for a corpectomy cage having a cavity according to certain embodiments of the present disclosure.

FIG. 7 is a diagram illustrating a perspective view of an exemplary prefabricated bone graft to be placed in a cavity according to certain embodiments of the present disclosure.

FIG. 8A is a diagram illustrating a perspective view of an exemplary central core of a corpectomy cage according to certain embodiments of the present disclosure.

FIG. 8B is a diagram illustrating a perspective view of an exemplary endcap to be fitted onto a corresponding corpectomy cage according to certain embodiments of the present disclosure.

FIG. 9 is a diagram illustrating a perspective view of an exemplary corpectomy cage according to certain embodiments of the present disclosure.

FIG. 10 is a diagram illustrating a perspective view of an exemplary corpectomy cage according to certain embodiments of the present disclosure.

FIG. 11 is a diagram illustrating a perspective view of an exemplary corpectomy cage according to certain embodiments of the present disclosure.

FIG. 12 is a flow chart illustrating an exemplary process for implanting a corpectomy cage according to certain embodiments of the present disclosure.

FIG. 13 is a flow chart illustrating an exemplary process for implanting a corpectomy cage according to certain embodiments of the present disclosure.

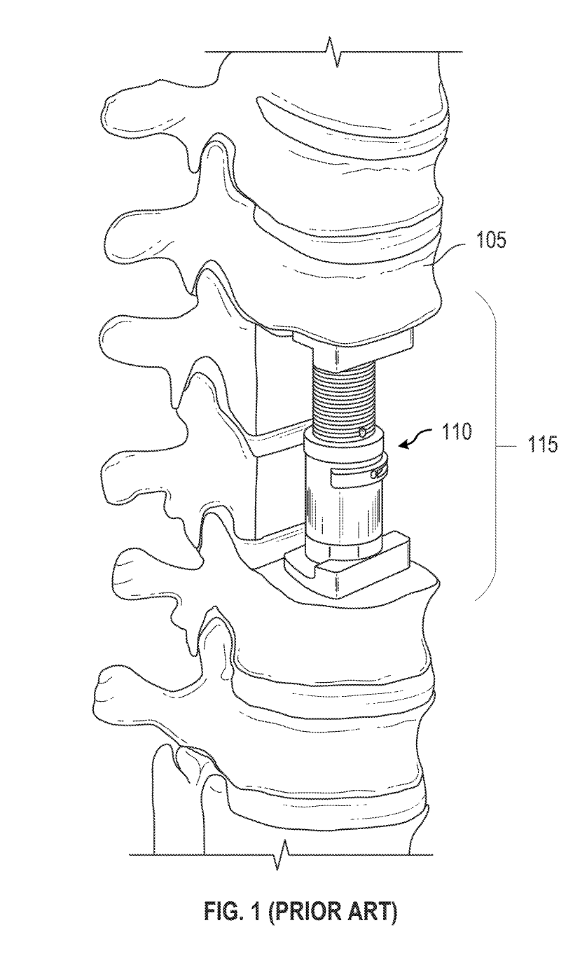

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION OF THE EMBODIMENTS

The following description is not to be taken in a limiting sense, but is made merely for the purpose of describing the general principles of the disclosed embodiments. The present embodiments address the problems described in the background while also addressing other additional problems as will be seen from the following detailed description. Numerous specific details are set forth to provide a full understanding of various aspects of the subject disclosure. It will be apparent, however, to one ordinarily skilled in the art that various aspects of the subject disclosure may be practiced without some of these specific details. In other instances, well-known structures and techniques have not been shown in detail to avoid unnecessarily obscuring the subject disclosure.

Some embodiments of the present disclosure provide a corpectomy cage to be implanted within a patient's spine. As disclosed herein, a corpectomy cage may include a central core with engageable endcaps configured to attach and to detach onto each ends of the central core. In other embodiments, the endcaps may be permanently affixed to each ends of the central core, thus eliminating any prior assembly requirements of the corpectomy cage by a medical personnel prior to implantation within a patient's spine. The corpectomy cage may then be implanted within the patient's intervertebral cavity space (e.g., a space in the spinal column created by removing one or more diseased or injured vertebra) such that the endcaps come in contact with the vertebra above and the vertebra below the intervertebral cavity space to restore the patient's collapsed vertebral column.

The central core of the corpectomy cage may come in various shapes and sizes so that the implanted corpectomy cage properly fits within the space where the injured or defected vertebra is now removed. By way of example only, the central core may be non-expandable or non-adjustable in height, but may be available in numerous different height lengths so as to enable a medical personnel or surgeon to choose the specific central core height that is best suited to accommodate the space within the patient's intervertebral cavity space. By way of another example, the central core in other embodiments may include a central core that expands and retracts in height so that a single central core is able to adjust and accommodate to a wide range of height lengths.

In some embodiments, the first end and second end of the core body may include a latching mechanism so that a user or surgeon may select a particular endcap shape or size to be connected or attached onto the central core of the corpectomy cage. The endcap of the corpectomy cage may be available in numerous different shapes, sizes and angles so that the selected endcap is suited to best accommodate the patient's spinal anatomy and curvature.

In some embodiments, the endcap may include at least one cavity so that bone material may be packed into the cavity space. Thus, when an endcap is placed in contact with the vertebra bone, the bone material in the cavity space may encourage the vertebra bone to fuse with the bone material. This may further encourage the likelihood of the surface of the endcap to more securely engage with the surface of the vertebra when the corpectomy cage is implanted within the patient's intervertebral cavity space, further ensuring a proper and stable placement of the corpectomy cage within the patient's spine.

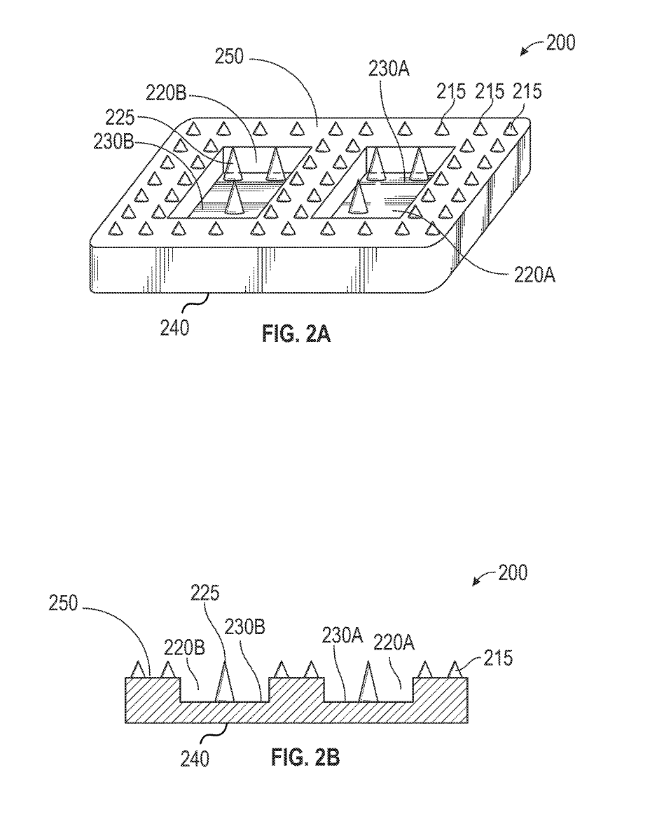

FIG. 2A is a diagram illustrating a perspective view of an exemplary endcap 200 for a corpectomy cage having cavities 220A, 220B according to certain embodiments of the present disclosure. FIG. 2B is a diagram illustrating a cross-sectional view of the same exemplary endcap 200 for a corpectomy cage according to certain embodiments of the present disclosure. As a result, aspects of FIGS. 2A and 2B will be described together. In the illustrated example, the endcap 200 has a first surface 240 configured to attach to a central core (not shown) of the corpectomy cage and a second surface 250 configured to come into contact with a surface of a vertebra above or below the intervertebral cavity space when implanted within a patient's spine. The endcap 200 may further be configured to come in a wide variety of shapes and sizes to accommodate different anatomy configurations of the vertebra unique to each patient. While exemplary FIGS. 2A and 2B illustrate an endcap 200 that is rectangular in shape, other shapes may be utilized. By way of example only, some shapes may include a circle, oval, trapezium, square, hexagon, etc. Thus a shape that best encompasses the shape and surface of the vertebra to be in contact with the endcap 200 may be selected. Additionally, the height or the thickness of the endcap 200 may include a range between 2-8 mm. By way of further example only, the endcap 200 may be made of material that includes titanium, peek plastic, carbon polymer, or stainless steel.

Furthermore, exemplary FIGS. 2A and 2B illustrate an endcap 200 with two cavities 220A, 220B formed within the endcap 200. Each cavity 220A or B may further include a corresponding cavity floor 230A or B configured to receive and contain bone material within the cavity. In some embodiments, the cavity 220A or B with a cavity floor 230A or B is packed with bone material to help promote the fusion and attachment of the vertebra with the endcap 200. The bone material may include the following material: bone allograft, bone autograft, osteoinductive agents, demineralized bone matrix, hydroxyapatite, etc., or a combination thereof. By packing and completely filling the cavity 220A or B with bone materials, the endcap 200 may then be specifically positioned to come in contact with a vertebra at one end of an intervertebral cavity space so that the bone material also comes in contact with the surface of the vertebra bone. By doing so, the bone material may fuse with the vertebra bone, thus further increasing the likelihood that the endcap is firmly and stably positioned with the corresponding surface of the vertebra.

In the instance where two or more cavities 220A, 220B are formed within the endcap 200, each cavity floor 230A or 230B may include varying shapes and heights to accommodate different amounts of bone material to be placed within the cavity 220A or B. By way of example only, the height of the cavity may include a range from 1-5 mm.

In some embodiments, the second surface 250 of the endcap 200 includes a raised surface 215 so as to become impacted into the vertebra bone and lessen any potential movement of the endcap when in contact the vertebra. Thus, the raised surface 215 may ensure that the endcap 200 is further securely engaged with the vertebra bone. The raised surface 215 may include varying shapes (e.g., teeth, spikes, mounds, circular or rectangular or triangular columns, saw-tooth edges, etc.) and sizes that may range between 1-5 mm in height.

In further embodiments, the endcap 200 further includes a raised surface 225 (e.g., teeth, spikes, mounds, circular or rectangular or triangular columns, saw-tooth edges, etc.) protruding from the cavity floor 230A or B. The teeth or pointed edges are configured to be sufficiently tall enough so as to also come in contact and become impacted into the vertebra. Again, for example, the impacting the raised surface 225 into the vertebra may further lessen any potential movement of the endcaps when in contact with the patient's vertebra.

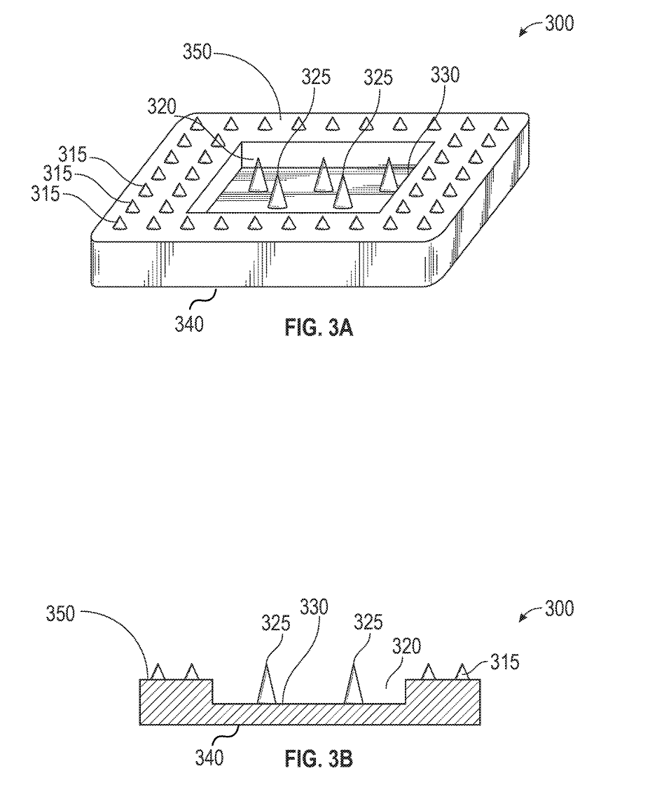

FIG. 3A is a diagram illustrating a perspective view of an exemplary endcap 300 for a corpectomy cage having a cavity 320 according to certain embodiments of the present disclosure. FIG. 3B is a diagram illustrating a cross-sectional view of the same exemplary endcap 300 for a corpectomy cage according to certain embodiments of the present disclosure. As a result, aspects of FIGS. 3A and 3B will be described together. In this particular embodiment, the endcap 300 has a first surface 340 configured to attach to a central core (not shown) of the corpectomy cage and a second surface 350 configured to come into contact with a surface of a vertebra above or below the intervertebral cavity space when implanted within a patient's spine. The second surface 350 includes a raised surface 315 that may include teeth or pointed edges to come in contact with a corresponding surface of a vertebra bone at one end of the intervertebral cavity space. The raised surface 315 may include varying shapes (e.g., teeth, spikes, mounds, circular or rectangular or triangular columns, saw-tooth edges, etc.). As such, the raised surface 315 may prevent or retard any potential movement of the endcap 300 when in contact with the surface of a vertebra.

In some embodiments, the endcap 300 may also include a cavity 320 with a cavity floor 330 configured to receive and contain bone material. The bone material may be selected to fuse with the vertebra bone so as to further ensure that the endcap 300 is securely attached and in continuous contact with the surface of the vertebra when implanted within the patient's intervertebral cavity space. In this particular embodiment, as illustrated in FIGS. 3A and 3B, the endcap 300 may include only one cavity 320. However, the number of cavities may range anywhere from 1-10 cavities. Furthermore, the cavity 320 may include a wide range of shapes and sizes in order to accommodate the appropriate number of cavities 320 and to accommodate various amounts of bone material. Additionally, the cavity floor 330 may further include a raised surface 325 with varying shapes and configurations that protrude from the cavity floor 330 so as to further prevent and retard any movement of the endcap 300 when in contact with the surface of the vertebra. Examples of such shapes may include teeth, spikes, mounds, circular or rectangular or triangular columns, saw-tooth edges, etc.

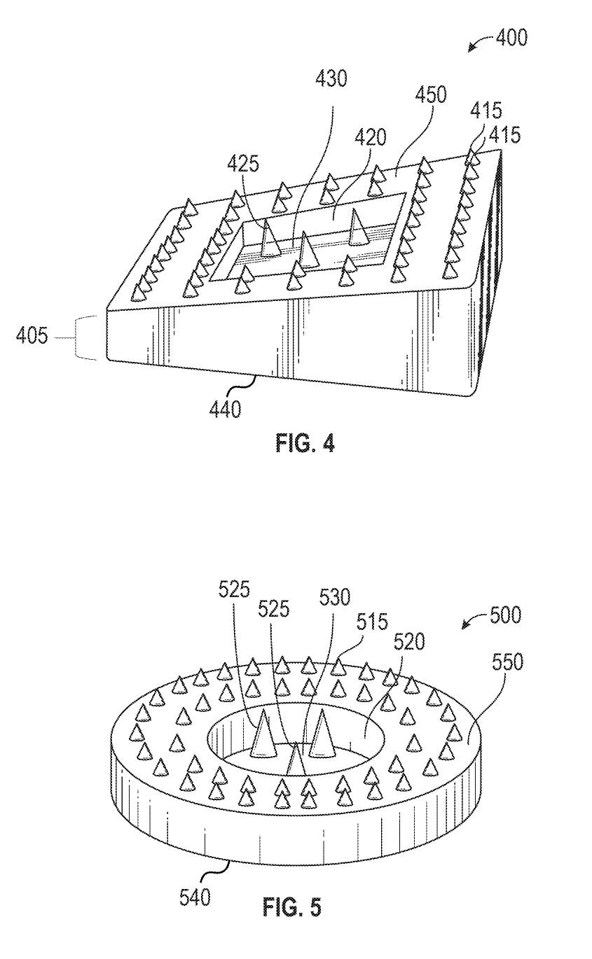

FIG. 4 is a diagram illustrating a perspective view of an exemplary endcap 400 for a corpectomy cage having a cavity 420 according to certain embodiments of the present disclosure. The endcap 400 has a first surface 440 configured to attach to a central core (not shown) of the corpectomy cage and a second surface 450 configured to come into contact with a surface of a vertebra above or below the intervertebral cavity space when implanted within a patient's spine. The second surface 450 may also include a lordotic angle 405 so as to create a wedge-like configuration. An endcap 400 with a lordotic angle 405 may be utilized to restore the natural inward curvature of a patient's spine when the endcap is implanted within the patient's intervertebral cavity space. The lordotic angle 405 of the endcap 400 may include a range from 1 to 11 degrees to accommodate varying spinal curvature degrees that are unique to each patient.

Additionally, the second surface 450 of the endcap 400 may further include a raised surface 415 (e.g., teeth, spikes, mounds, circular or rectangular or triangular columns, saw-tooth edges, etc.) so as to impact into the vertebra bone and lessen any potential movement of the endcap 400 when implanted within the patient's spine. Furthermore, the endcap 400 may also include a cavity 420 with a cavity floor 430 so as to receive and contain bone material within the endcap 400. In some embodiments, the cavity floor 430 may further include a raised surface 425 (e.g., teeth, spikes, mounds, circular or rectangular or triangular columns, saw-tooth edges, etc.) to further retard movement of the endcap 400 when the endcap 400 is in contact with the vertebra bone.

FIG. 5 is a diagram illustrating a perspective view of an exemplary endcap 500 having a cavity 520 for a corpectomy cage according to certain embodiments of the present disclosure. The endcap 500 has a first surface 540 configured to attach to a central core (not shown) of the corpectomy cage and a second surface 450 configured to come into contact with a surface of a vertebra above or below the intervertebral cavity space when implanted within a patient's spine. In this particular example, the endcap 500 has a circular shape. The configuration of the circular shape of the endcap 500 may be best suited to accommodate the particular vertebra shape and size. However, as discussed above, the endcap 500 is not limited to a circular shape, and may include other shapes that may be better suited to securely fit and best match the shape, size, and/or anatomy of the vertebra of interest.

In this particular embodiment, the endcap 500 may include a raised surface 515 (e.g., teeth, spikes, mounds, circular or rectangular or triangular columns, saw-tooth edges, etc.) to retard movement of the endcap 500 when in contact with the corresponding vertebra bone of the patient's intervertebral cavity space. The endcap 500 may also include a cavity 520 with a cavity floor 530 to be filled with bone material to be fused with the vertebra bone. In the instance where the bone material contained in the cavity 520 properly fuses with the vertebra bone, there is a greater likelihood that the endcap 500 is securely in fixed contact and attachment with the corresponding vertebra.

Additionally, the cavity floor 530 may also include a raised surface 525 (e.g., teeth, spikes, mounds, circular or rectangular or triangular columns, saw-tooth edges, etc.) protruding from the cavity floor 530 to further aid in retarding any potential movement of the endcap 500 when in contact with the corresponding vertebra bone of the patient's intervertebral cavity space.

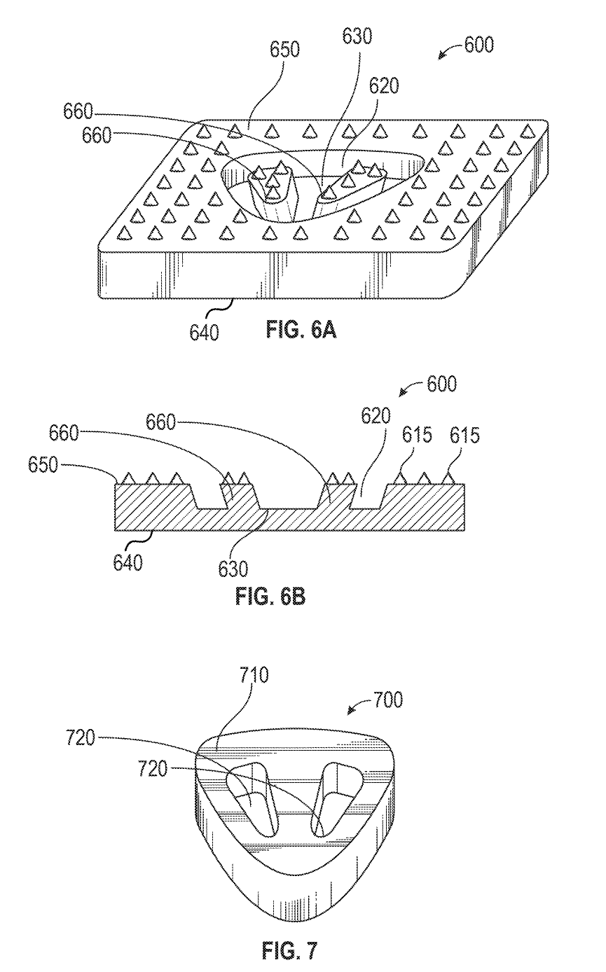

FIG. 6A is a diagram illustrating a perspective view of an exemplary endcap 600 for a corpectomy cage having a cavity 620 according to certain embodiments of the present disclosure. FIG. 6B is a diagram illustrating a cross-sectional view of the same exemplary endcap 600 for a corpectomy cage according to certain embodiments of the present disclosure. As such, aspects of FIGS. 6A and 6B will be described together. The endcap 600 has a first surface 640 configured to attach to a central core (not shown) of the corpectomy cage and a second surface 650 configured to come into contact with a surface of a vertebra above or below the intervertebral cavity space when implanted within a patient's spine. In this particular embodiment, the cavity 620 may be configured to receive bone material. In some embodiments, the bone material may include a pre-assembled bone graft 700 that is molded to fit and be disposed within the cavity 620, as illustrated in FIG. 7. The pre-assembled bone graft 700 may already include pre-packed bone material 710 already arranged and packed tightly within a molded configuration. The pre-packed bone material 710 may be configured to help promote the fusion and attachment of the vertebra with the endcap 600. The pre-packed bone material 710 may include bone allograft, bone autograft, osteoinductive agents, demineralized bone matrix, hydroxyapatite, or a combination thereof.

Because the pre-assembled bone graft 700 is already pre-packed and pre-assembled into a molded configuration, this prevents any spilling or the need to manually pack and load the bone material into the cavity 605. As such, the cavity 620 in FIG. 6A may be shaped and configured to receive the corresponding pre-assembled bone graft 700.

While FIG. 6A illustrates a cavity 620 having a particular shape that corresponds to the shape of the pre-assembled bone graft 700, it should be noted that the shape of the cavity 620 and the shape of the pre-assembled bone graft 700 may be configured to any shape best suited to fit within the endcap. Thus, in order to ensure that the pre-assembled bone graft 700 is properly fitted within the cavity 620, the shape of the cavity 620 may mimic and correspond with the shape of the pre-assembled bone graft 700 to be fitted within the cavity 620. Additionally, in some embodiments, the cavity 620 may include a cavity floor 630 so as to properly receive and contain the pre-assembled bone graft 700 within the cavity 620. For example, as illustrated in embodiments FIGS. 6A, 6B, and 7, the endcap 600 includes two islands 660 protruding from the cavity floor 630 that are configured to mate with two corresponding openings 720 formed in the pre-assembled bone graft 700. However, it should be noted that the cavity 620 and the pre-assembled bone graft 700 may come in varying shapes with or without islands 660 protruding from the cavity floor 630.

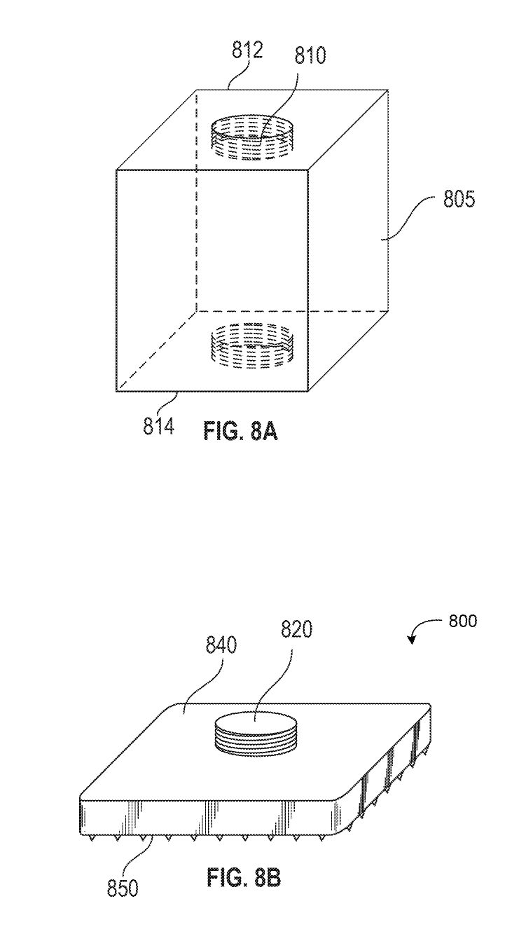

FIG. 8A illustrates a perspective view of an exemplary central core 805 of a corpectomy cage and FIG. 8B illustrates an exemplary endcap 800 to be fitted onto the exemplary central core 805 according to certain embodiments of the present disclosure. As such, aspects of FIGS. 8A and 8B will be described together.

In this particular embodiment, the central core 805 may include a first end 812 and a second end 814, such that an endcap 800 may attach onto each end of the central core 805. In some embodiments, the endcap 800 attached to the central core 805 includes a first surface 840 configured to be attached to the first end 812 or the second end 814 of the central core 805. In further embodiments, the endcap 800 to be attached to the first end 812 or the second end 814 of the central core 805 includes a second surface 850 configured to be in contact with the vertebra above or the vertebra below the patient's intervertebral cavity space.

In order to securely attach the endcap 800 to a corresponding end of the central core 805, such as the first end 812 or the second end 814, the endcap 800 may include a locking mechanism 820 that couples or latches onto the corresponding locking portion or mechanism 810 located on the central core 805. In some embodiments, the endcap 800 includes a locking mechanism 820 with a female/male fastener, female/male screw threads, or a snap fastener that couples onto the corresponding locking portion or mechanism 810 on the central core 805. As such, the central core 805 may also include a corresponding locking portion or mechanism 810 that may include the corresponding female/male fastener, female/male screw threads, or a snap fastener. However, it should be noted that any type of secure fastening mechanisms that allows the endcap 800 to be securely attached or locked onto the central core 805 may be utilized. In some embodiments, the central core 805 may be made of material including titanium, peek plastic, carbon polymer, or stainless steel.

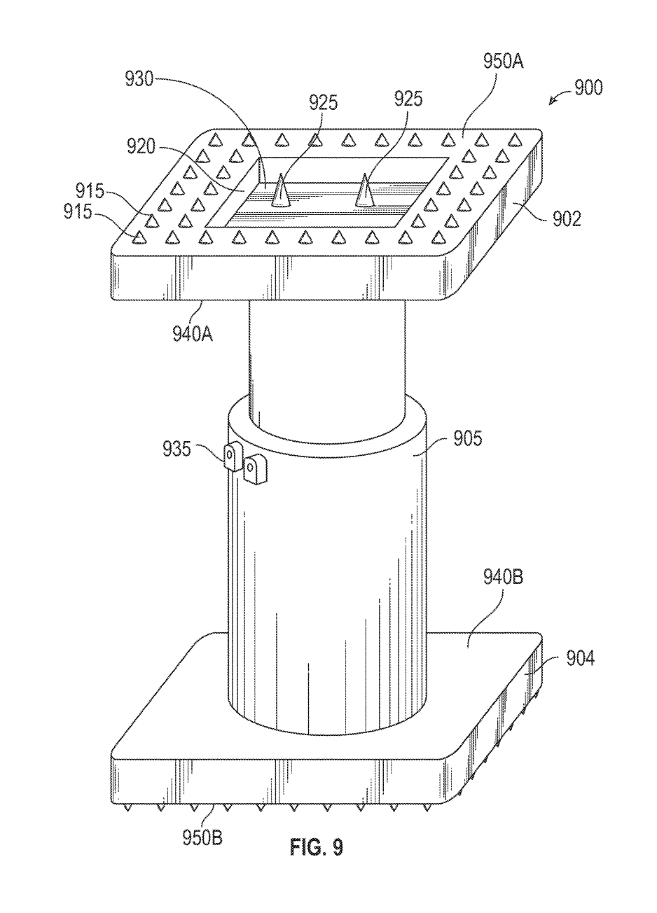

FIG. 9 is a diagram illustrating a perspective view of an exemplary corpectomy cage 900 according to certain embodiments of the present disclosure. As illustrated, there is a central core 905 with two endcaps 902, 904 located at each respective ends of the central core 905. The endcaps 902, 904 may be already affixed to the central core 905, so that any pre-assembly requirements by a medical personnel or a surgeon are not required prior to the corpectomy procedure. In other instances, the endcaps 902, 904 may be engageably attached and/or detached from the central core 905, thus allowing a medical personnel or surgeon to try various different endcap 902, 904 combinations and the option to manually assemble the corpectomy cage 900 prior to the corpectomy procedure.

The endcaps 902, 904 may include a first surface 940A and B configured to attach onto each end of the central core 905 of the corpectomy cage 900 and a second surface 950A and B configured to come into contact with the surface of a vertebra. The second surface 950 A and B may include a raised surface 915 with varying shapes (e.g., teeth, spikes, mounds, circular or rectangular or triangular columns, saw-tooth edges, etc.) so as to help lessen any potential movement of the endcaps 902, 904 when in contact with the vertebra.

In some embodiments, the endcaps 902, 904 attached to the central core 905 may also include a cavity 920 configured to contain bone material that will fuse with the surface of the vertebra. Additionally, the cavity 920 may also include a cavity floor 930 so as to effectively receive and contain the bone material to be fused with the vertebra. In some embodiments, by way of example only, the cavity floor 910 may also include a raised surface 925 that may include teeth, spikes, mounds, circular or rectangular or triangular columns, saw-tooth edges, etc. so as to further prevent and retard any movement of the endcap 902, 904 when in contact with the surface of the vertebra. It also should be noted that the two endcaps 902, 904 attached to the ends of the central core 905 need not be the same endcap 902, 904 shape, size, or configuration. Instead, the two endcaps 902,904 may each be of different size, shape, and configuration that best matches and accommodates the anatomy of the corresponding vertebra the endcap is selected to be in contact with. As such, two different endcaps 915, 930 may be affixed or attached to the ends of the central core 905.

The central core 905 may have a height that ranges from 15-70 mm. In this particular embodiment, the central core 905 may be adjustable in height such that the height of the central core 905 may extended or retracted. The central core 905 may include an adjusting mechanism 935 attached to the side of the central core 905 to aid in raising and lowering the height of the central core that is best suited to fit within the patient's intervertebral cavity space. Additionally, the adjusting mechanism 935 may also include a lock so as to ensure that the selected height of the central core 905 is maintained in a locked position.

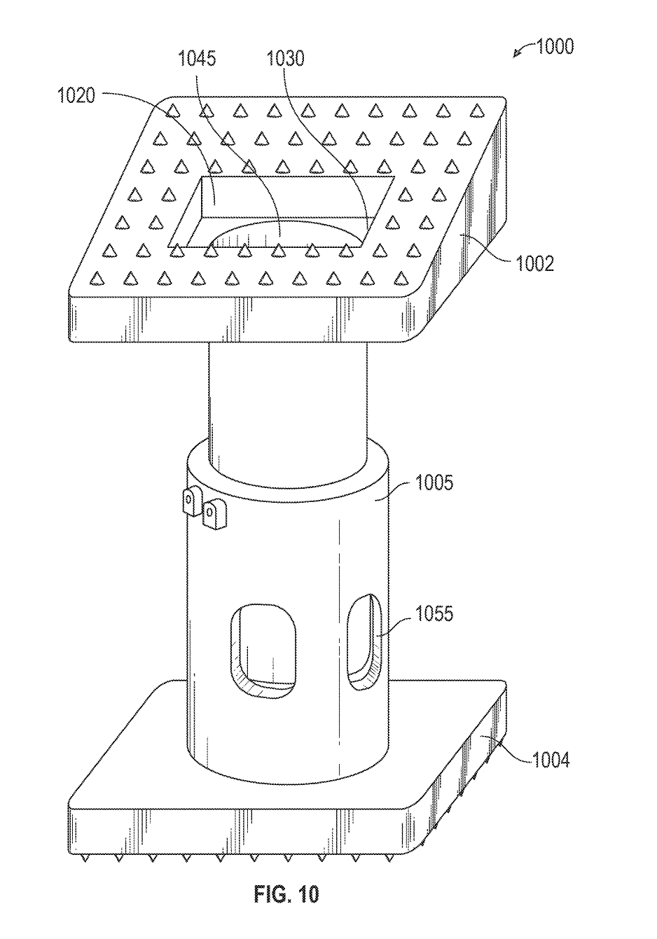

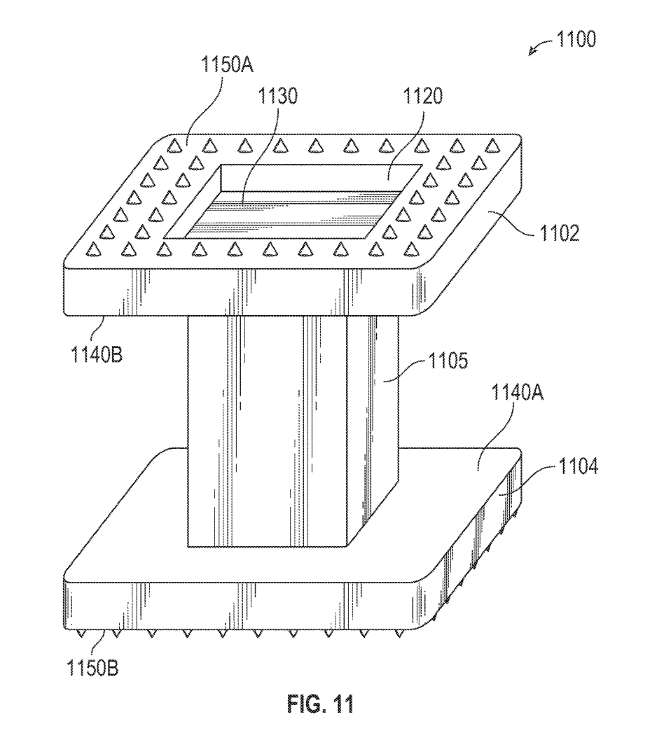

FIG. 10 is a diagram illustrating a perspective view of an exemplary corpectomy cage 1000 according to certain embodiments of the present disclosure. In this particular embodiment, the central core 1005 may include a hollow center. Additionally, the central core 1005 may include window openings 1055 on the side of the central core 1005 to allow bone material to be manually packed into the central core 1005 via insertion of the bone material through the window openings 1055. However, in other embodiments, the central core 1005 may be a solid body (not shown here) such that bone material or any other material cannot be filled within the central core 1020.

As further illustrated in FIG. 10, endcaps 1002,1004 may be attached to each corresponding end of the central core 1005. The endcaps 1002, 1004 may be identical in shape, size, and configuration, or may also be two completely different sizes, shapes, and configurations, as illustrated in FIG. 10. The appropriate endcaps 1002, 1004 to be attached to the ends of the central core 1020 may depend on the curvature, shape, and anatomy of the corresponding surface of the vertebra bone to be in contact with the endcap 1002, 1004 surface.

In the instance the central core 1020 contains a hollow body, the endcaps 1002, 1004 may include a cavity 1020 with a cavity floor 1030 that has an opening 1045 to allow the bone material to be pass through the opening 1045 of the cavity floor 1030 and directly into the hollow central core 1005. In some embodiments, the area of the opening 1045 within the cavity floor 1045 may range from 30-80% of the area of the cavity floor 1045 space.

As such, in this particular embodiment, bone material may completely fill the hollow central core 1005 and even completely fill the cavity 1020 space. At such an event, there is essentially a single column of bone material packed within the corpectomy cage 1000. Thus when the corpectomy cage 1000 filled with a column of bone material is implanted within the patient's intervertebral cavity space, the vertebra above and below the intervertebral cavity space may fuse with the bone material available on the surface of the endcap 1002, 1004 of the filled cavity 1020. When the vertebra fuses with the bone material at each end of the endcaps 1002, 1004, a single solid fusion of the patient's vertebral column is successfully formed.

FIG. 11 is a diagram illustrating a perspective view of an exemplary corpectomy cage 1100 according to certain embodiments of the present disclosure. In this particular embodiment, the corpectomy cage 1100 may include a central core 1105 that is not adjustable by height. Instead, different embodiments may include a central core 1105 with varying different heights to be selected from.

In some embodiments, the endcaps 1102, 1104 have a first surface 1140 A and B configured to attach to a central core 1105 of the corpectomy cage 1100 and a second surface 1150 A and B configured to come in contact with a surface of the vertebra. In some embodiments, the endcaps 1102, 1104 may be affixed or detachable from the central core 1105. Additionally, in the instance that the central core 1105 has a solid core, each ends of the central core 1105 may act and provide a floor 1130 to the cavity 1120 formed within each endcap 1102, 1104. Because the solid core of the central core 1105 may provide itself as a cavity floor 1130, bone material may be contained within the cavity 1120. In some instances, the locking portion (not shown here) of the central core 1105 is located at each respective ends of the central core 1105 where the corresponding endcaps 1102, 1104 are to be attached. As such, the locking portion (not shown here) of the central core 1105 may also provide itself as the floor 1130 to the cavity 1120 of the endcap, thus allowing bone material to be contained within the cavity 1120 when the locking portion (not shown here) of the endcap 1102, 1104 is coupled to the corresponding locking portion (not shown here) of the central core 1105.

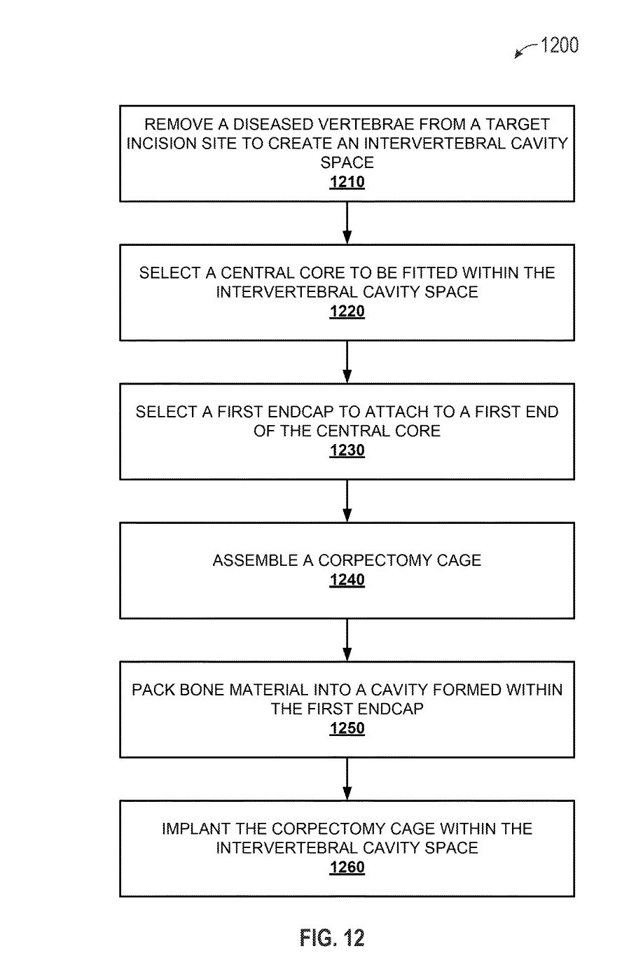

FIG. 12 is a flow chart illustrating an exemplary process 1200 for implanting a corpectomy cage according to certain embodiments of the present disclosure. The exemplary process begins at operation 1210 by removing a diseased vertebra of a patient from a target incision site to create an intervertebral cavity space within a patient's vertebral column. The exemplary process 1200 may then proceed to operation 1220, where a doctor or any other medical personnel may select a central core with a height that is best suited to be securely fitted within the intervertebral cavity space. Ideally, the height of the central core and the corpectomy cage implanted within the intervertebral cavity space will restore the natural height of the patient's vertebral column. By way of example only, the central core may have a height with a range from 15 to 70 mm.

The exemplary process proceeds to 1230, where the first endcap is selected to attach to an end of the central core. The first endcap may be selected that best matches the patient's existing spinal configuration and anatomy, thus allowing a proper fit between the first endcap and the surface of the vertebra when the corpectomy cage is implanted within the patient's intervertebral cavity. In some embodiments, a second endcap may be attached to the second end of the central core, such that the selected second endcap is also one that is best suited to match the patient's spinal anatomy, such that there is a proper fit between the second endcap and the surface of the corresponding vertebra. As such, when selecting the first and/or second endcap to be attached to the central core of the corpectomy cage, consideration of the natural inward lordotic curvature of the spine may also be considered.

Next, exemplary process 1200 may then proceed to operation 1240, where the corpectomy cage may be assembled by attaching the first endcap to the first end of the central core. In some embodiments, the corpectomy cage may be assembled by further attaching the second endcap to the second end of the central core. The exemplary process 1200 may further proceed to operation 1250, where bone material is packed into a cavity formed within the first endcap. By way of example only, the bone material may include bone allograft, bone autograft, osteoinductive agents, demineralized bone matrix, hydroxyapatite, or a combination thereof.

The cavity may include a cavity floor so as to effectively receive and contain the bone material within the cavity. In some embodiments, the cavity floor has an opening, such that the opening includes 30-80% of the area of the cavity floor space. As such, bone material may pass through the opening of the cavity floor in the instance where the endcap with a cavity floor opening is attached to a central core that is hollow. Additionally, the bone material may completely fill the cavity space so as to reach the top surface layer of the endcap, thus allowing the bone material to come in contact with the surface of the vertebra when the corpectomy cage is implanted within the patient's intervertebral cavity space. Next, the exemplary process 1200 proceeds to operation 1260, where the corpectomy cage is implanted within the intervertebral cavity space, such that the bone material placed within the cavity allows for a proper fusion between the bone material and the surface of the vertebra above or below the intervertebral cavity space.

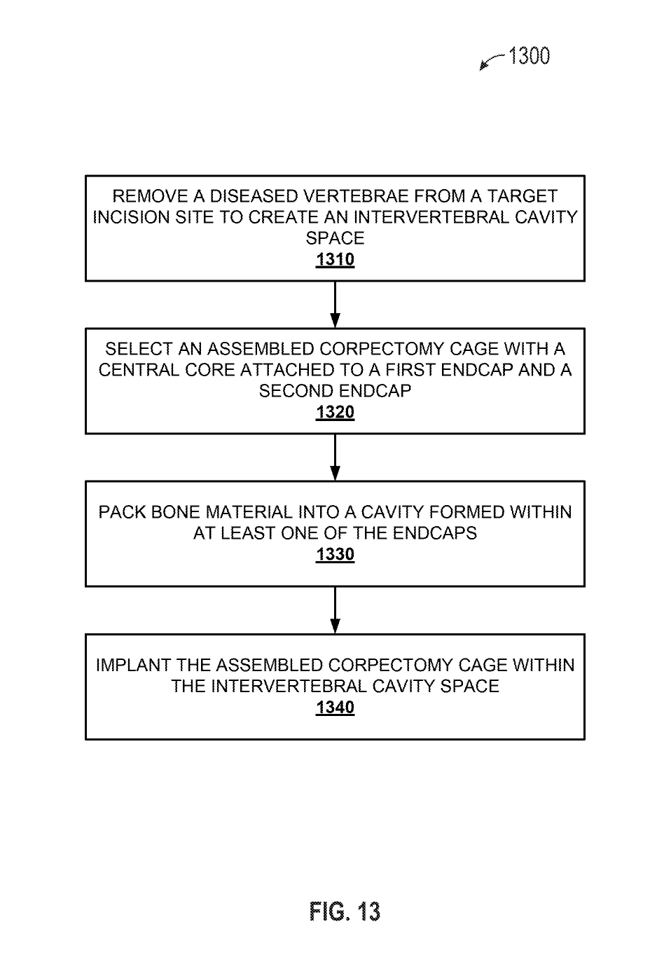

FIG. 13 is a flow chart illustrating an exemplary process 1300 for implanting a corpectomy cage according to certain embodiments of the present disclosure. The exemplary process 1300 for implanting an already assembled corpectomy cage begins at operation 1310 by removing a diseased vertebra from a target incision site to create an intervertebral cavity space within the patient's vertebral column. The exemplary process 1300 then proceeds to operation 1320, where a corpectomy cage with an already attached central core and endcaps is selected to best fit within the patient's intervertebral cavity space. Additionally, the selected corpectomy cage may be selected that includes a first endcap and a second endcap that is suited to best fit the anatomy and surface of the corresponding vertebra to make contact with the endcaps of the corpectomy cage.

The exemplary process 1300 proceeds to operation 1330, where bone material is packed into a cavity formed within at least one of the first endcap and the second endcap, with the cavity having a cavity floor to contain and receive the bone material. The bone material may include at least one of bone allograft, bone autograft, osteoinductive agents, demineralized bone matrix, and hydroxyapatite so as to help fuse with the surface of the vertebra upon contact.

The exemplary process 1300 thus proceeds to operation 1340 where the assembled corpectomy cage is implanted within the intervertebral cavity space, such that the first endcap makes contact with a first vertebra above the intervertebral cavity and the second endcap make contact with a second vertebra below the intervertebral cavity space. In some embodiments, the corpectomy cage includes a central core that is adjustable in height. As such, the height of the assembled corpectomy cage may be adjusted prior to implanting the corpectomy cage within the intervertebral cavity space. In other instances, the height of the assembled corpectomy cage may be adjusted after implanting the corpectomy cage within the intervertebral cavity space.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

1. A method for implanting a corpectomy cage comprising:
removing a diseased vertebra of a patient from a target incision site to create an intervertebral cavity space within a vertebral column;
selecting a central core with a height suited to be securely fitted within the intervertebral cavity space to restore the height of the vertebral column;
selecting a first endcap that matches the patient's existing spinal configuration and anatomy to attach to a first end of the central core;
assembling the corpectomy cage by attaching a first surface of the first endcap to the first end of the central core;
packing a bone material into a cavity formed within the first endcap, the cavity having a cavity floor to contain the bone material; and
implanting the corpectomy cage within the intervertebral cavity space to promote a fusion between the bone material and a vertebra at one end of the intervertebral cavity space.

2. The method of claim 1 further comprising selecting a second endcap to attach to a second end of the central core that matches the patient's existing spinal configuration and anatomy.

3. The method of claim 2, wherein selecting the first endcap and the second endcap comprises selecting an endcap with a lordotic angle configured to restore a natural inward lordotic curvature of a spine.

4. The method of claim 3, wherein the lordotic angle of the endcap has a range from 1 to 11 degrees.

5. The method of claim 1, wherein the first endcap has a second surface configured to engage with the vertebra at one end of the intervertebral cavity space, the first endcap comprising a raised surface protruding from the second surface and configured to retard a movement of the first endcap when in contact with the vertebra at the one end of the intervertebral cavity space.

6. The method of claim 1, wherein the raised surface protruding from the cavity floor comprises at least one of teeth, pointed edges, spikes, mounds, circular columns, rectangular columns, triangular columns, and saw tooth edges.

7. The method of claim 1, wherein the first endcap comprises a snap fit attachment located at the first surface, such that the first surface of the first endcap securely interlocks with a corresponding snap fit attachment on the first end of the central core.

8. The method of claim 1, wherein the bone material comprises at least one of bone allograft, bone autograft, osteoinductive agents, demineralized bone matrix, and hydroxyapatite.

9. The method of claim 1, wherein the central core has a solid body.

10. The method of claim 9, wherein the cavity comprises an opening that passes through the height of the endcap, and an end of the central core with the solid body is configured to provide a floor to the opening, thereby allowing the cavity to contain the bone material.

11. The method of claim 1, wherein the central core comprises a hollow body configured to be packed with bone material.

12. The method of claim 11, further comprising packing the bone material into the hollow body until both the hollow body and the cavity are completely filled to promote a solid single union of the vertebral column.

13. The method of claim 11, wherein the bone material is packed into the hollow body via an opening in the cavity floor.

14. The method of claim 13, wherein an area of the opening in the cavity floor ranges from 30-80% of an area of the cavity floor.

15. A method for implanting an assembled corpectomy cage comprising:
removing a diseased vertebra from a target incision site to create an intervertebral cavity space within a vertebral column;
selecting the assembled corpectomy cage comprising a central core, a first endcap attached to a first end of the central core, and a second endcap attached to a second end of the central core;
packing a bone material into a cavity formed within at least one of the first endcap and the second endcap, the cavity having a cavity floor; and
implanting the assembled corpectomy cage within the intervertebral cavity space such that the first endcap makes a contact with a first vertebra above the intervertebral cavity space, and the second endcap makes contact with a second vertebra below the intervertebral cavity space.

16. The method of claim 15, further comprising adjusting a height of the assembled corpectomy cage prior to implanting the assembled corpectomy cage within the intervertebral cavity space.

17. The method of claim 15, further comprising adjusting a height of the assembled corpectomy cage after implanting the assembled corpectomy cage within the intervertebral cavity space.

18. The method of claim 17, wherein adjusting the height of the central core comprises using an adjusting mechanism comprises a screw on the central core to raise or lower the height of the central core.

19. The method of claim 15, wherein the central core has a solid body.

20. The method of claim 15, wherein the central core has a hollow body configured to be packed with the bone material.

21. The method of claim 20, wherein packing the bone material into the cavity comprises filling the hollow body with the bone material until both the hollow body and the cavity is completely filled with the bone material to promote a single solid fusion of the vertebral column.

22. The method of claim 15, wherein the first endcap comprises a first surface configured to attach to the central core of the corpectomy cage and a second surface configured to engage with the first vertebra.

23. The method of claim 22, wherein the first endcap has a raised surface comprising at least one of teeth, pointed edges, spikes, mounds, circular columns, rectangular columns, triangular columns, and saw tooth edges to retard a movement of the first endcap when in contact with the vertebra.

24. The method of claim 15, wherein the at least one of the first endcap and the second endcap comprises a raised surface protruding from the cavity floor to come in contact with a corresponding one of the first vertebra and the second vertebra.
